(19) United States
(12) Patent Application Publication
(10) Pub. No.: US 2026/0240991 A1
(43) Pub. Date: Aug. 20, 2026

(54) METHODS OF HEMATOPOIETIC PRECURSOR CELLS MOBILIZATION

(71) Applicant: BioLineRx Ltd., ModiIn (IL)

(72) Inventor: Gideon STEIN, Rosh HaAyin (IL)

(73) Assignee: BioLineRx Ltd., ModiIn (IL)

(21) Appl. No.: 19/469,422

(22) PCT Filed: Mar. 26, 2024
(86) PCT No.: PCT/IL2024/050309

§ 371 Date: Sep. 26, 2025

Related U.S. Application Data
(60) Provisional application No. 63/454,684, filed on Mar. 26, 2023.

(51) Int. Cl. A61K 39/395; A61K 31/395; A61K 38/12

(52) U.S. Cl. CPC A61K 39/3955; A61K 31/395; A61K 38/12

(57) ABSTRACT

A method of mobilizing hematopoietic precursor cells from the bone marrow to the peripheral blood of a subject is provided. The method comprising (a) administering to the subject an effective amount of natalizumab; and (b) administering to the subject an effective amount of a CXCR4 inhibitor, wherein step (b) is effected at least 48 hours following step (a). Also provided are uses of such mobilization procedures in cell collection and in treatment.

### RELATED APPLICATION/S

This application claims the benefit of priority of U.S. Provisional Patent Application No. 63/454,684, filed on Mar. 26, 2023, the contents of which are incorporated herein by reference in their entirety.

### SEQUENCE LISTING STATEMENT

The XML, entitled 99455.xml, created on 25 Mar. 2024, comprising 215,618 bytes, submitted concurrently with the filing of this application is incorporated herein by reference.

### FIELD AND BACKGROUND OF THE INVENTION

The present invention, in some embodiments thereof, relates to methods of hematopoietic precursor cells mobilization.

Hematopoietic cell transplantation is an important and often life-saving treatment for many hematological malignancies and solid tumors as means of reconstitution of blood cells following high dose chemotherapy. The Gold standard for mobilization typically includes use of granulocyte colony stimulating factor (G-CSF)-mobilized peripheral blood stem cells (PBSC) that has largely replaced bone marrow (BM) as a source of stem cells for both autologous and allogeneic cell transplantation. In spite of the increased number of CD34+ stem cells obtained after G-CSF mobilization compared to BM harvests, one still needs to obtain a minimum number of CD34/kg (~≥2×10⁶) to ensure successful and consistent multi-lineage engraftment and sustained hematopoietic recovery. Conversely, SDF-1/CXCR4 interaction plays a key role in HSC quiescence and retention within the bone marrow. Plerixafor (Mozobil, AMD3100) is a bicyclam molecule which reversibly inhibits SDF-1 binding to CXCR4, promoting HSC mobilization, Plerixafor is approved to be used in combination with G-CSF for stem cell mobilization in patients with myeloma and lymphoma.

Motixafortide (4F-benzoyl-TN14003; Biokine Therapeutics, Rehovit, Israel) (T-140) also known as BKT 140, is a highly selective CXCR4 antagonist, originally designed to inhibit binding of the human immunodeficiency virus (HIV) to CXCR4. In mice, this agent induced up to a 10-fold increase in PB progenitor cells, which peaked at 1-2 hours following the administration of the dose. BKT 140 synergized with G-CSF, leading to a 78-fold increase in PB progenitor cells over controls, higher than seen with a combination of Plerixafor and G-CSF (Abraham et al. Stem Cells. 2007; 25:2158-66), In a phase I/IIA dose-escalation trial in myeloma patients, BTK 140 was well-tolerated and resulted in a dose-dependent increase in the mean absolute PB CD34+ cells. The highest dose tested (900 μg/kg) resulted in a mean PB CD34+ count of 20.6×10⁶/kg, reducing the number of apheresis sessions required from 2.25 with lower doses to 1 (Nagler et al. ASH Annual Meeting Abstracts; 2010. p. 2260).

Integrins are a structurally and functionally diverse family of transmembrane glycoproteins that mediate cell-cell and cell-matrix interactions in a wide range of biological contexts. Eighteen different α and eight different β subunits exist in vertebrates, giving rise to 24 different non-covalently-bound αβ heterodimers, which are able to bind a wide variety of ligands. One such heterodimer expressed in hematopoietic stem cells, α₄β₁, termed very late antigen 4(VLA-4), mediates HSC adhesion to vascular cell adhesion molecule-1 (VCAM-1) within the bone marrow stroma. In preclinical studies, administration of anti-VLA-4 antibodies resulted in mobilization of HSC progenitors into the bloodstream (Vermeulen et al. Blood. 1998; 92:894-900).

Natalizumab, a recombinant humanized monoclonal antibody against d4 subunit of VLA-4, approved for treatment of multiple sclerosis (MS) and Crohn's disease, has been found to increase peripheral blood CD34+ cells in patients with relapsing-remitting MS. Zohren et al showed a gradual increase in the circulating CD34+ cells in MS patients, with a maximal concentration of 10.4 CD34+ cells/μL 72 hours following administration of Natalizumab (Zohren et al. Blood. 2008; 111:3893-5). Jing et al demonstrated a 7-fold increase in PB CD34+ cells and a 7-fold, dose-dependent increase in BM CD34+ cells in patients with MS treated with Natalizumab, with a maximum absolute count reached on day 4 following treatment (Jing et al. 2010; 45:1489-96). Moreover, concurrent VLA-4 and CXCR4 blockade has been shown to have a greater than an additive effect in stem cell mobilization in primates, when compared with either agent alone (Bonig et al. Stem Cells. 2009; 27:836-7). Unfortunately, Natalizumab-induced elevation in PB CD34+ cells persists at least 1 month following administration of the drug, which limits its use in healthy donors (Zohren supra and Bonig supra).

Despite many developments there is still a need for novel therapeutic modalities that will lead to the most effective and efficient stem cell mobilization strategies, especially in those patients who are at risk for mobilization failure.

Additional background art includes:
U.S. Pat. No. 8,455,450
WO 2018/085574
PMID: 24476957

### SUMMARY OF THE INVENTION

According to an aspect of some embodiments of the present invention there is provided a method of mobilizing hematopoietic precursor cells from the bone marrow to the peripheral blood of a subject, comprising:
(a) administering to the subject an effective amount of natalizumab; and
(b) administering to the subject an effective amount of a CXCR4 inhibitor, wherein step (b) is effected at least 48 hours following step (a).

According to an aspect of some embodiments of the present invention there is provided a method for obtaining a therapeutically effective amount of hematopoietic precursor cells from a subject, comprising:
(a) administering to the subject an effective amount of natalizumab;
(b) administering to the subject an effective amount of a CXCR4 inhibitor, wherein step (b) is effected at least 48 hours following step (a); and
(c) harvesting the hematopoietic precursor cells by apheresis.

According to an aspect of some embodiments of the present invention there is provided an effective amount of a combination of natalizumab and a CXCR4 inhibitor for use in mobilization of hematopoietic precursor cells in a subject, wherein the CXCR4 inhibitor is administered to the subject at least 48 hours following administration of the natalizumab.

According to an aspect of some embodiments of the present invention there is provided an effective amount of a combination of natalizumab and a CXCR4 inhibitor for use in a method of harvesting hematopoietic precursor cells by apheresis comprising mobilizing the hematopoietic precursor cells, wherein the hematopoietic precursor cells are subsequently harvested by the apheresis, wherein the CXCR4 inhibitor is administered to the subject at least 48 hours following administration of the natalizumab.

According to some embodiments of the invention, the subject is a human subject.

According to some embodiments of the invention, the subject is a donor of the hematopoietic precursor cells.

According to some embodiments of the invention, the subject is diagnosed with cancer.

According to some embodiments of the invention, the hematopoietic precursor cells are for autologous transplantation.

According to some embodiments of the invention, the hematopoietic precursor cells are for allogeneic transplantation.

According to some embodiments of the invention, the effective amount comprises repetitive cycles of administration of the natalizumab and the CXCR4 inhibitor.

According to some embodiments of the invention, the at least 48 hours comprises up to 96 hours.

According to some embodiments of the invention, the at least 48 hours comprises up to 72 hours.

According to some embodiments of the invention, the CXCR4 inhibitor is a peptide, a small molecule, an antibody, a nucleic acid or a combination of same.

According to some embodiments of the invention, the CXCR4 inhibitor is a peptide as set forth in SEQ ID NO: 1.

According to some embodiments of the invention, the CXCR4 inhibitor is a small molecule.

According to some embodiments of the invention, the CXCR4 inhibitor is AMD3100.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### DESCRIPTION OF SPECIFIC EMBODIMENTS OF THE INVENTION

The present invention, in some embodiments thereof, relates to methods of hematopoietic precursor cells mobilization.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventor has unexpectedly uncovered that timing the administration of CXCR4 inhibitor for at least 48 hours following natalizumab administration allows obtaining substantially high amounts of hematopoietic precursor cells.

Thus, according to an aspect of the invention, there is provided a method of mobilizing hematopoietic precursor cells from the bone marrow to the peripheral blood of a subject, comprising:
(a) administering to the subject an effective amount of natalizumab; and
(b) administering to the subject an effective amount of a CXCR4 inhibitor, wherein step (b) is effected at least 48 hours following step (a).

According to an additional or an alternative aspect there is provided a method for obtaining a therapeutically effective amount of hematopoietic precursor cells from a subject, comprising:
(a) administering to the subject an effective amount of natalizumab;
(b) administering to the subject an effective amount of a CXCR4 inhibitor, wherein step (b) is effected at least 48 hours following step (a); and
(c) harvesting the hematopoietic precursor cells by apheresis.

According to an additional or an alternative aspect there is provided an effective amount of a combination of natalizumab and a CXCR4 inhibitor for use in mobilization of hematopoietic precursor cells in a subject, wherein the CXCR4 inhibitor is administered to the subject at least 48 hours following administration of the natalizumab.

According to an additional or an alternative aspect there is provided an effective amount of a combination of natalizumab and a CXCR4 inhibitor for use in a method of harvesting hematopoietic precursor cells by apheresis comprising mobilizing the hematopoietic precursor cells, wherein the hematopoietic precursor cells are subsequently harvested by the apheresis, wherein the CXCR4 inhibitor is administered to the subject at least 48 hours following administration of the natalizumab.

As used herein, the term “hematopoietic precursor cells” refers to a fraction of cells that can be found in the bone marrow (BM) niche. These cells are characterized by multipotentiality, enabling them to self-renew and also to produce mature blood cells, such as erythrocytes, leukocytes, platelets, and lymphocytes. These cells typically include hematopoietic stem cells (HSC) that express CD34 and are referred to CD34+ cells. CD34 is a marker of human HSC, and all colony-forming activity of human bone marrow (BM) cells is found in the CD34+ fraction.

Hematopoietic precursor cells in their native form or modified (e.g., differentiated in culture, expanded in culture) following apheresis are typically used in the treatment of a variety of medical conditions.

As used herein the term “mobilization” refers to the release of hematopoietic precursor cells (e.g., hematopoietic stem cells) from bone marrow into peripheral blood circulation.

As used herein “increasing mobilization” refers to inducing mobilization of peripheral blood precursor cells, to elevate circulating levels of HSCs, or to enhance or facilitate hematopoietic reconstitution or engraftment, in a subject in need thereof.

According to one embodiment, mobilization of hematopoietic stem cells is increased by at least about 10%, 20%, 30%, 40%, 50%, 60%, 70%, 80%, 90%, 100%, 120%, 150% or 200% as compared to mobilization in the absence of the present agents (i.e., CXCR4 inhibitor and natalizumab). According to one embodiment, mobilization of hematopoietic precursor cells is increased by at least about 2 fold, 3 fold, 4 fold, 5 fold, 6 fold, 7 fold, 8 fold, 9 fold or 10 fold as compared to mobilization in the absence of the present agents. Measuring enhancement of hematopoietic precursor cell mobilization is known to one of ordinary skill in the art.

As used herein the term “subject” refers to a mammalian subject e.g., a human subject, of any gender or age. The subject may be a healthy subject who serves as a donor for hematopoietic precursor cells transplantation (e.g. also referred to as a ‘donor subject’). Alternatively, the subject may suffer from a disease or condition (e.g. malignant disease or non-malignant disease such as an immune deficiency) and hence is in need of stem cell mobilization or stem cell transplantation (i.e., autologous; or from a donor i.e., non-autologous i.e., allogeneic). In the latter case the subject is a recipient in need of a stem cell transplant.

The term “healthy subject” as used herein refers to a subject who has not been diagnosed with a disease or disorder amenable to treatment by hematopoietic precursor cells transplantation. According to a specific embodiment, the healthy subject does not suffer from a hematopoietic disorder or malignancy.

As used herein, the term “CXCR4 inhibitor” refers to molecules and compositions that interfere with or inhibit the biological activity of the CXCR4 receptor. Biological activity of the CXCR4 receptor can include mobilization of stem cells to the blood as well as entry of the virus to the cell or replication of the virus in the cell.

The CXCR4 inhibitors can encompass numerous classes of chemical molecules, e.g., small organic or inorganic molecules, polysaccharides, biological macromolecules, e.g., peptides, proteins, peptide analogs and derivatives, peptidomimetics, antibodies, antibody fragments, nucleic acids, nucleic acid analogs and derivatives such as aptamers, an extract made from biological materials such as bacteria, plants, fungi, or animal cells or tissues, naturally occurring or synthetic compositions.

Without wishing to be bound by a theory, a CXCR4 inhibitor can act by a number of different pathways. For example, a CXCR4 inhibitor can bind to a ligand bind site on the CXCR4 receptor and interfere with binding of the ligand to the CXCR4 receptor, bind to a nonligand binding site on the CXCR4 receptor and interfere with binding of the ligand to the CXCR 4 receptor, bind with a CXCR4 receptor ligand and interfere with binding of the ligand to the CXCR4 receptor, or inhibit the expression of a polynucleotide (e.g., mRNA) expressing CXCR4

In some embodiments, a CXCR4 inhibitor inhibits the biological activity of the CXCR4 receptor by at least 5%, at least 10%, at least 15%, at least 20%, at least 30%, at least 40%, at least 50%, at least 60%, at least 70%, at least 75%, at least 80%, at least 85%, at least 90%, or at least 95% relative to a control. In some embodiments, a CXCR4 inhibitor completely abrogates the biological activity of the CXCR4 receptor relative to a control. A control can comprise a sample that is not treated an inhibitor.

In some embodiments, a CXCR4 inhibitor is a nucleic acid. Exemplary CXCR4 nucleic acid inhibitors include, but are not limited to, antisense oligonucleotides, siRNAs, shRNAs, microRNAs, aptamers, ribozymes and decoy oligonucleotides. A CXCR4 nucleic acid inhibitor can inhibit the expression of a CXCR4 gene.

Exemplary anti CXCR4 siRNAs are described, for example, in U.S. Pat. App. Pub. No. 2007/0238868, No. 2009/0253772, content of both of which is incorporated herein by reference. Some exemplary CXCR4 antisense oligonucleotides are described, for example, in U.S. Pat. App. Pub. No. 2004/0209837, content of which is incorporated herein by reference.

In some embodiments, the CXCR4 inhibitor binds to CXCR4 or to CXCL12 (SDF-1 alpha). In another embodiment, the CXCR4 inhibitor is an antibody or antibody fragment. In some embodiments, the CXCR4 inhibitor is a small molecule, for example, AMD-3100, ALX40-4C, T22, T140, Met-SDFlbeta, T134, or AMD-3465.

Exemplary CXCR4 inhibitors include, but are not limited to, 2,2′-bicyclam; 6,6′-bicyclam; the embodiments set forth in U.S. Pat. Nos. 5,021,409, and 6,001,826, and in particular 1,1′-[1,4-phenylene-bis(methylene)]-bis-1,4,8,11tetraazacyclotetradecane, set forth in U.S. Pat. No. 5,583,131, and designated herein AMD3100. In some embodiments, a CXCR4 inhibitor can be N′-(1 Hbenzimidazol-2-yl methyl)-N′-(5,6,7,8-tetrahydroquinoline8-yl)-butane-1,4-diamine as described in U.S. Patent Publication No. 2003/0220341, CTCF-0214; CTCF-9908; CP-1221 (linear peptides, cyclic peptides, natural amino-acids, unnatural amino acids, and peptidomimetic compounds); 4F-benzoylTN24003; KRH-1120; KRH-1636; KRH-2731; polyphemusin analogue; ALX40-4C; or those described in WO 01/85196; WO 99/50461; WO 01/94420; WO 03/090512, each of which is incorporated by reference herein in its entirety.

In some embodiments, CXCR4 inhibitors include the T-140 analogs and antibodies described in US Patent Publication 2010/0055088, the cycle polyamines described in US Patent Publication 2009/0221683, and the compounds disclosed in US Patent Publication Nos. 2004/0209921, 2005/0059702, 2005/0043367, 2005/0277670, 2010/0178271, and 2003/0220341; U.S. Pat. Nos. 5,021,409, 6,001,826, 5,583,131, and Patent Publication WO 03/011277, each of which are incorporated herein by reference in their entirety.

CXCR4 inhibitors can also include, but are not limited to, polypeptides that specifically bind to CXCR4. Such inhibitors include T140 and derivatives of T140. Exemplary derivatives of T140 include, but are not limited to, TN14003, TC14012, and TE14011 as well as those found in Tamamura, H. et al. Org. Biomol. Chem. 1:3656-3662, 2003, which is incorporated by reference herein in its entirety.

According to specific embodiments, the CXCR4-antagonistic peptides of the present invention are for example, 4F-benzoyl-TN14003 (SEQ ID NO: 1) analogs and derivatives and are structurally and functionally related to the peptides disclosed in patent applications WO 2002/020561 and WO 2004/020462, also known as “T-140 analogs”, as detailed hereinbelow.

In various particular embodiments, the T-140 analog or derivative has an amino acid sequence as set forth in the following formula (I) or a salt thereof:

(I)
1 2 3 4 5 6 7 8 9 10 11 12 13 14
A₁-A₂-A₃-Cys-Tyr-A₄-A₅-A₆-A₇-A₈-A₉-A₁₀-Cys-A₁₁

wherein:
A₁ is an arginine, lysine, ornithine, citrulline, alanine or glutamic acid residue or a N-α-substituted derivative of these amino acids, or A₁ is absent;
A₂ represents an arginine or glutamic acid residue if A₁ is present, or A₂ represents an arginine or glutamic acid residue or a N-α-substituted derivative of these amino acids if A₁ is absent;
A₃ represents an aromatic amino acid residue;
A₄, A₅ and A₉ each independently represents an arginine, lysine, ornithine, citrulline, alanine or glutamic acid residue;
A₆ represents a proline, glycine, ornithine, lysine, alanine, citrulline, arginine or glutamic acid residue;
A₇ represents a proline, glycine, ornithine, lysine, alanine, citrulline or arginine residue;
A₈ represents a tyrosine, phenylalanine, alanine, naphthylalanine, citrulline or glutamic acid residue;
A₁₀ represents a citrulline, glutamic acid, arginine or lysine residue;
A₁₁ represents an arginine, glutamic acid, lysine or citrulline residue wherein the C-terminal carboxyl may be derivatized;
and the cysteine residue of the 4-position or the 13-position can form a disulfide bond, and the amino acids can be of either L or D form.

Exemplary peptides according to formula (I) are peptides having an amino acid sequence as set forth in any one of SEQ ID NOS: 1-72, as presented in Table 1 hereinbelow.

TABLE 1
T-140 and currently preferred T-140 analogs

| | SEQ | |
|---|---|---|
| | ID | |
| Analog | NO: | Amino acid sequence |
| 4F-benzoyl- | 1 | 4F-benzoyl-Arg-Arg-Nal-Cys-Tyr-Cit-Lys-DLys-Pro-Tyr-Arg-Cit- |
| TN14003 | | Cys-Arg-NH₂ |
| AcTC14003 | 2 | Ac-Arg-Arg-Nal-Cys-Tyr-Cit-Lys-DLys-Pro-Tyr-Arg-Cit-Cys-Arg- |
| | | OH |
| AcTC14005 | 3 | Ac-Arg-Arg-Nal-Cys-Tyr-Arg-Lys-DCit-Pro-Tyr-Arg-Cit-Cys-Arg- |
| | | OH |
| AcTC14011 | 4 | Ac-Arg-Arg-Nal-Cys-Tyr-Cit-Lys-DCit-Pro-Tyr-Arg-Cit-Cys-Arg-OH |
| AcTC14013 | 5 | Ac-Arg-Arg-Nal-Cys-Tyr-Cit-Lys-DLys-Pro-Tyr-Cit-Cit-Cys-Arg-OH |
| AcTC14015 | 6 | Ac-Cit-Arg-Nal-Cys-Tyr-Cit-Lys-DLys-Pro-Tyr-Arg-Cit-Cys-Arg-OH |
| AcTC14017 | 7 | Ac-Cit-Arg-Nal-Cys-Tyr-Arg-Lys-DCit-Pro-Tyr-Arg-Cit-Cys-Arg-OH |
| AcTC14019 | 8 | Ac-Arg-Arg-Nal-Cys-Tyr-Arg-Lys-DCit-Pro-Tyr-Cit-Cit-Cys-Arg-OH |
| AcTC14021 | 9 | Ac-Cit-Arg-Nal-Cys-Tyr-Arg-Lys-DLys-Pro-Tyr-Cit-Cit-Cys-Arg-OH |
| AcTC14012 | 10 | Ac-Arg-Arg-Nal-Cys-Tyr-Cit-Lys-DCit-Pro-Tyr-Arg-Cit-Cys-Arg- |
| | | NH₂ |
| AcTC14014 | 11 | Ac-Arg-Arg-Nal-Cys-Tyr-Cit-Lys-DLys-Pro-Tyr-Cit-Cit-Cys-Arg- |
| | | NH₂ |
| AcTC14016 | 12 | Ac-Cit-Arg-Nal-Cys-Tyr-Cit-Lys-DLys-Pro-Tyr-Arg-Cit-Cys-Arg- |
| | | NH₂ |
| AcTC14018 | 13 | Ac-Cit-Arg-Nal-Cys-Tyr-Arg-Lys-DCit-Pro-Tyr-Arg-Cit-Cys-Arg- |
| | | NH₂ |
| AcTC14020 | 14 | Ac-Arg-Arg-Nal-Cys-Tyr-Arg-Lys-DCit-Pro-Tyr-Cit-Cit-Cys-Arg- |
| | | NH₂ |
| AcTC14022 | 15 | Ac-Cit-Arg-Nal-Cys-Tyr-Arg-Lys-DLys-Pro-Tyr-Cit-Cit-Cys-Arg- |
| | | NH₂ |
| TE14001 | 16 | H-DGlu-Arg-Nal-Cys-Tyr-Arg-Lys-DLys-Pro-Tyr-Arg-Cit-Cys-Arg- |
| | | OH |
| TE14002 | 17 | H-Arg-Glu-Nal-Cys-Tyr-Arg-Lys-DLys-Pro-Tyr-Arg-Cit-Cys-Arg-OH |
| TE14003 | 18 | H-Arg-Arg-Nal-Cys-Tyr-Glu-Lys-DLys-Pro-Tyr-Arg-Cit-Cys-Arg-OH |
| TE14004 | 19 | H-Arg-Arg-Nal-Cys-Tyr-Arg-Glu-DLys-Pro-Tyr-Arg-Cit-Cys-Arg-OH |
| TE14005 | 20 | H-Arg-Arg-Nal-Cys-Tyr-Arg-Lys-DGlu-Pro-Tyr-Arg-Cit-Cys-Arg-OH |
| TE14006 | 21 | H-Arg-Arg-Nal-Cys-Tyr-Arg-Lys-DLys-Pro-Tyr-Glu-Cit-Cys-Arg-OH |
| TE14007 | 22 | H-Arg-Arg-Nal-Cys-Tyr-Arg-Lys-DLys-Pro-Tyr-Arg-Cit-Cys-Glu-OH |
| TE14011 | 23 | H-Arg-Arg-Nal-Cys-Tyr-Cit-Lys-DGlu-Pro-Tyr-Arg-Cit-Cys-Arg-NH₂ |
| TE14012 | 24 | H-Arg-Arg-Nal-Cys-Tyr-DGlu-Lys-DCit-Pro-Tyr-Arg-Cit-Cys-Arg- |
| | | NH₂ |
| TE14013 | 25 | H-Arg-Arg-Nal-Cys-Tyr-DGlu-Lys-DGlu-Pro-Tyr-Arg-Cit-Cys-Arg- |
| | | NH₂ |
| TE14014 | 26 | H-DGlu-Arg-Nal-Cys-Tyr-Cit-Lys-DGlu-Pro-Tyr-Arg-Cit-Cys-Arg- |
| | | NH₂ |
| TE14015 | 27 | H-Arg-Arg-Nal-Cys-Tyr-Cit-Lys-DGlu-Pro-DGlu-Arg-Cit-Cys-Arg- |
| | | NH₂ |
| TE14016 | 28 | H-Arg-Arg-Nal-Cys-Tyr-Cit-Lys-DGlu-Pro-Tyr-Arg-DGlu-Cys-Arg- |
| | | NH₂ |
| AcTE14014 | 29 | Ac-DGlu-Arg-Nal-Cys-Tyr-Cit-Lys-DGlu-Pro-Tyr-Arg-Cit-Cys-Arg- |
| | | NH₂ |
| AcTE14015 | 30 | Ac-Arg-Arg-Nal-Cys-Tyr-Cit-Lys-DGlu-Pro-DGlu-Arg-Cit-Cys-Arg- |
| | | NH₂ |
| AcTE14016 | 31 | Ac-Arg-Arg-Nal-Cys-Tyr-Cit-Lys-DGlu-Pro-Tyr-Arg-DGlu-Cys-Arg- |
| | | NH₂ |
| TF1: | 32 | Ac-Arg-Arg-Nal-Cys-Tyr-Cit-Lys-DGlu-Pro-Tyr-Arg-Cit-Cys-Arg- |
| AcTE14011 | | NH₂ |
| TF2: guanyl- | 33 | guanyl-Arg-Arg-Nal-Cys-Tyr-Cit-Lys-DGlu-Pro-Tyr-Arg-Cit-Cys- |
| TE14011 | | Arg-NH₂ |
| TF3: | 34 | TMguany1-Arg-Arg-Nal-Cys-Tyr-Cit-Lys-DGlu-Pro-Tyr-Arg-Cit-Cys- |
| TMguanyl- | | Arg-NH₂ |
| TE14011 | | |
| TF4: | 35 | TMguanyl-Arg-Nal-Cys-Tyr-Cit-Lys-DGlu-Pro-Tyr-Arg-Cit-Cys-Arg- |
| TMguany1- | | NH₂ |
| TE14011 (2- | | |
| 14) | | |
| TF5: 4F- | 36 | 4F-benzoyl-Arg-Arg-Nal-Cys-Tyr-Cit-Lys-DGlu-Pro-Tyr-Arg-Cit- |
| benzoyl- | | Cys-Arg-NH₂ |
| TE14011 | | |
| TF6: 2F- | 37 | ₂F-benzoyl-Arg-Arg-Nal-Cys-Tyr-Cit-Lys-DGlu-Pro-Tyr-Arg-Cit- |
| benzoyl- | | Cys-Arg-NH₂ |
| TE14011 | | |
| TF7: APA- | 38 | APA-Arg-Nal-Cys-Tyr-Cit-Lys-DGlu-Pro-Tyr-Arg-Cit-Cys-Arg-NH₂ |
| TE14011 (2- | | |
| 14) | | |
| TF8: | 39 | desamino-R-Arg-Nal-Cys-Tyr-Cit-Lys-DGlu-Pro-Tyr-Arg-Cit-Cys- |
| desamino-R- | | Arg-NH₂ |
| TE14011 (2- | | |
| 14) | | |
| TF9: guanyl- | 40 | Guanyl-Arg-Nal-Cys-Tyr-Cit-Lys-DGlu-Pro-Tyr-Arg-Cit-Cys-Arg- |
| TE14011 (2- | | NH₂ |
| 14) | | |
| TF10: | 41 | succinyl-Arg-Nal-Cys-Tyr-Cit-Lys-DGlu-Pro-Tyr-Arg-Cit-Cys-Arg- |
| succinyl- | | NH₂ |
| TE14011 (2- | | |
| 14) | | |
| TF11: | 42 | glutaryl-Arg-Nal-Cys-Tyr-Cit-Lys-DGlu-Pro-Tyr-Arg-Cit-Cys-Arg- |
| glutaryl- | | NH₂ |
| TE14011 (2- | | |
| 14) | | |
| TF12: | 43 | deaminoTMG-APA-Arg-Nal-Cys-Tyr-Cit-Lys-DGlu-Pro-Tyr-Arg-Cit- |
| deaminoTM | | Cys-Arg-NH₂ |
| G-APA- | | |
| TE14011 (2- | | |
| 14) | | |
| TF15: H-Arg- | 44 | R-CH₂-Arg-Nal-Cys-Tyr-Cit-Lys-DGlu-Pro-Tyr-Arg-Cit-Cys-Arg- |
| CH2NH- | | NH₂ |
| RTE14011 | | |
| (2-14) | | |
| TF17: | 45 | H-Arg-Nal-Cys-Tyr-Cit-Lys-DGlu-Pro-Tyr-Arg-Cit-Cys-Arg-NH₂ |
| TE14011 | | |
| (2-14) | | |
| TF18: | 46 | TMguanyl-Arg-Arg-Nal-Cys-Tyr-Cit-Lys-DCit-Pro-Tyr-Arg-Cit-Cys- |
| TMguany1- | | Arg-NH₂ |
| TC14012 | | |
| TF19: ACA- | 47 | ACA-Arg-Arg-Nal-Cys-Tyr-Cit-Lys-DCit-Pro-Tyr-Arg-Cit-Cys-Arg- |
| TC14012 | | NH₂ |
| TF20: ACA- | 48 | ACA-Arg-Arg-Nal-Cys-Tyr-Arg-Lys-DLys-Pro-Tyr-Arg-Cit-Cys-Arg- |
| T140 | | OH |
| TZ14011 | 49 | H-Arg-Arg-Nal-Cys-Tyr-Cit-Arg-DLys-Pro-Tyr-Arg-Cit-Cys-Arg- |
| | | NH₂ |
| AcTZ14011 | 50 | Ac-Arg-Arg-Nal-Cys-Tyr-Cit-Arg-DLys-Pro-Tyr-Arg-Cit-Cys-Arg- |
| | | NH₂ |
| AcTN14003 | 51 | Ac-Arg-Arg-Nal-Cys-Tyr-Cit-Lys-DLys-Pro-Tyr-Arg-Cit-Cys-Arg- |
| | | NH₂ |
| AcTN14005 | 52 | Ac-Arg-Arg-Nal-Cys-Tyr-Arg-Lys-DCit-Pro-Tyr-Arg-Cit-Cys-Arg- |
| | | NH₂ |
| 4F-benzoyl- | 53 | 4F-benzoyl-Arg-Arg-Nal-Cys-Tyr-Cit-Lys-DGlu-Pro-Tyr-Arg-Cit- |
| TN14011-Me | | Cys-Arg-NHMe |
| 4F-benzoyl- | 54 | 4F-benzoyl-Arg-Arg-Nal-Cys-Tyr-Cit-Lys-DGlu-Pro-Tyr-Arg-Cit- |
| TN14011-Et | | Cys-Arg-NHEt |
| 4F-benzoyl- | 55 | 4F-benzoyl-Arg-Arg-Nal-Cys-Tyr-Cit-Lys-DGlu-Pro-Tyr-Arg-Cit- |
| TN14011-iPr | | Cys-Arg-NHiPr |
| 4F-benzoyl- | 56 | 4F-benzoyl-Arg-Arg-Nal-Cys-Tyr-Cit-Lys-DGlu-Pro-Tyr-Arg-Cit- |
| TN14011- | | Cys-Arg-tyramine |
| tyramine | | |
| TA14001 | 57 | H-Ala-Arg-Nal-Cys-Tyr-Arg-Lys-DLys-Pro-Tyr-Arg-Cit-Cys-Arg-OH |
| TA14005 | 58 | H-Arg-Arg-Nal-Cys-Tyr-Ala-Lys-DLys-Pro-Tyr-Arg-Cit-Cys-Arg-OH |
| TA14006 | 59 | H-Arg-Arg-Nal-Cys-Tyr-Arg-Ala-DLys-Pro-Tyr-Arg-Cit-Cys-Arg-OH |
| TA14007 | 60 | H-Arg-Arg-Nal-Cys-Tyr-Arg-Lys-DAla-Pro-Tyr-Arg-Cit-Cys-Arg-OH |
| TA14008 | 61 | H-Arg-Arg-Nal-Cys-Tyr-Arg-Lys-DLys-Ala-Tyr-Arg-Cit-Cys-Arg- |
| | | OH |
| TA14009 | 62 | H-Arg-Arg-Nal-Cys-Tyr-Arg-Lys-DLys-Pro-Ala-Arg-Cit-Cys-Arg-OH |
| TA14010 | 63 | H-Arg-Arg-Nal-Cys-Tyr-Arg-Lys-DLys-Pro-Tyr-Ala-Cit-Cys-Arg-OH |
| TC14001 | 64 | H-Cit-Arg-Nal-Cys-Tyr-Arg-Lys-DLys-Pro-Tyr-Arg-Cit-Cys-Arg-OH |
| TC14003 | 65 | H-Arg-Arg-Nal-Cys-Tyr-Cit-Lys-DLys-Pro-Tyr-Arg-Cit-Cys-Arg-OH |
| TN14003 | 66 | H-Arg-Arg-Nal-Cys-Tyr-Cit-Lys-DLys-Pro-Tyr-Arg-Cit-Cys-Arg-NH₂ |
| TC14004 | 67 | H-Arg-Arg-Nal-Cys-Tyr-Arg-Cit-DLys-Pro-Tyr-Arg-Cit-Cys-Arg-OH |
| TC14012 | 68 | H-Arg-Arg-Nal-Cys-Tyr-Cit-Lys-DCit-Pro-Tyr-Arg-Cit-Cys-Arg-NH₂ |
| T-140 | 69 | H-Arg-Arg-Nal-Cys-Tyr-Arg-Lys-DLys-Pro-Tyr-Arg-Cit-Cys-Arg-OH |
| TC14011 | 70 | H-Arg-Arg-Nal-Cys-Tyr-Cit-Lys-DCit-Pro-Tyr-Arg-Cit-Cys-Arg-OH |
| TC14005 | 71 | H-Arg-Arg-Nal-Cys-Tyr-Arg-Lys-DCit-Pro-Tyr-Arg-Cit-Cys-Arg-OH |
| TC14018 | 72 | H-Cit-Arg-Nal-Cys-Tyr-Arg-Lys-DCit-Pro-Tyr-Arg-Cit-Cys-Arg-NH₂ |

According to a specific embodiment, in each one of SEQ ID NOS: 1-72, two cysteine residues are coupled in a disulfide bond.

In another embodiment, the analog or derivative has an amino acid sequence as set forth in SEQ ID NO:65 (H-Arg-Arg-Nal-Cys-Tyr-Cit-Lys-DLys-Pro-Tyr-Arg-Cit-Cys-Arg-OH; TC14003).

In another embodiment, the peptide used in the compositions and methods of the invention consists essentially of an amino acid sequence as set forth in SEQ ID NO:1. In another embodiment, the peptide used in the compositions and methods of the invention comprises an amino acid sequence as set forth in SEQ ID NO:1. In another embodiment, the peptide is at least 60%, at least 70% or at least 80% homologous to SEQ ID NO:1. In another embodiment, the peptide is at least 90% homologous to SEQ ID NO:1. In another embodiment, the peptide is at least about 95% homologous to SEQ ID NO:1. Each possibility represents a separate embodiment of the present invention.

In various other embodiments, the peptide is selected from SEQ ID NOS: 1-72, wherein each possibility represents a separate embodiment of the present invention.

In another embodiment, the peptide has an amino acid sequence as set forth in any one of SEQ ID NOS: 1-4, 10, 46, 47, 51-56, 65, 66, 68, 70 and 71. In another embodiment, the peptide has an amino acid sequence as set forth in any one of SEQ ID NOS: 4, 10, 46, 47, 68 and 70. In another embodiment, the peptide has an amino acid sequence as set forth in any one of SEQ ID NOS: 1, 2, 51, 65 and 66. In another embodiment, the peptide has an amino acid sequence as set forth in any one of SEQ ID NOS: 53-56.

In an embodiment, the peptide has an amino acid sequence as set forth in SEQ ID NO:1. In another embodiment, the peptide has an amino acid sequence as set forth in SEQ ID NO:2. In another embodiment, the peptide has an amino acid sequence as set forth in SEQ ID NO:51. In another embodiment, the peptide has an amino acid sequence as set forth in SEQ ID NO:66.

Other CXCR4 peptide inhibitors (antagonists) include but are not limited to LY2510924 (by Lilly Oncology), CTCE-9908 (Huang et al. 2009 Journal of Surgical Research 155:231-236), Fc131 analogs and nanobodies as specified in the citations below (each of which is incorporated herein by reference in its entirety):

Tan N C, Yu P, Kwon Y-U, Kodadek T. High-throughput evaluation of relative cell permeability between peptoids and peptides. Bioorg Med Chem. 2008; 16:5853-61.

Kwon Y-U, Kodadek T. Quantitative evaluation of the relative cell permeability of peptoids and peptides. J Am Chem Soc. 2007; 129:1508.

Miller S, Simon R, Ng S, Zuckermann R, Kerr J, Moos W. Comparison of the proteolytic susceptibilities of homologous L-amino acid, D-amino acid, and N-substituted glycine peptide and peptoid oligomers. Drug Dev Res. 1995; 35:20-32.

Yoshikawa Y, Kobayashi K, Oishi S, Fujii N, Furuya T. Molecular modeling study of cyclic pentapeptide CXCR4 antagonists: new insight into CXCR4-FC131 interactions. Bioorg Med Chem Lett. 2012; 22:2146-50.

Jaähnichen S, Blanchetot C, Maussang D, Gonzalez-Pajuelo M, Chow K Y, Bosch L, De Vrieze S, Serruys B, Ulrichts H, Vandevelde W. CXCR4 nanobodies (VHH-based single variable domains) potently inhibit chemotaxis and HIV-1 replication and mobilize stem cells. Proc Natl Acad Sci USA. 2010; 107:20565-70.

According to a specific embodiment, the peptide has an amino acid sequence as set forth in SEQ ID NO: 1 under the name Motixafortide.

As mentioned, the VLA-4 inhibitory (VLA4i) agent is Natalizumab [abbreviated herein as (N)].
According so some embodiment, the Natalizumab is sold under the brand name Tysabri®.
According so some embodiment, the Natalizumab is sold under the brand name Antegren®.

According to specific embodiments the peptide (e.g., BL-8040) of the invention or the pharmaceutical composition comprising same is administered in a dose ranging between 0.1 to 10 mg/kg of body weight, between 0.1 to 2 mg/kg of body weight, between 0.1 to 1 mg/kg of body weight, between 0.3 to 10 mg/kg of body weight, between 0.3 to 2.

According to a specific embodiment, BL-8040 is administered at a dose of 1-2 mg/kg body weight.

According to a specific embodiment, the, BL-8040 is administered at a dose of 1.25-1.5 mg/kg body weight.

According to a specific embodiment, the BL-8040 is administered at a dose of 1.25 mg/kg body weight.

According to a specific embodiment, the BL-8040 is administered subcutaneously (SC).

As mentioned, the CXCR4 inhibitor (e.g., BL-8040) is administered at a time point of at least 48 hours following administration of said Natalizumab.

According to a specific embodiment, the at least 48 hours comprises up to 96 hours (e.g., 48-96 hours).

According to a specific embodiment, the at least 48 hours comprises up to 80 hours (e.g., 48-80 hours).

According to a specific embodiment, the at least 48 hours comprises up to 72 hours (e.g., 48-72 hours).

According to a specific embodiment, the at least 48 hours comprises about 48 hours (e.g., 48 hours).

According to a specific embodiment, the method comprises performing apheresis to retrieve the hematopoietic precursor cells from peripheral blood of the subject following administering the agents.

According to some embodiments of the invention, about 4 hours and up to about 12 hours, following administration of the CXCR4 inhibitor, the subject undergoes apheresis.

Methods of collecting peripheral blood are well known in the art and include, but not limited to drawing of (e.g., up to 500 ml) whole blood from the subject and collection in a container containing an anti-coagulant (e.g. heparin or citrate); and apheresis.

As used herein, the term “apheresis” refers to a procedure in which the peripheral blood of an individual is passed through an apparatus, yielding a predominant constituent (e.g. HSCs), and returning the other constituents to the subject's circulation. Apheresis is in general a three-step process comprising: (1) withdrawing blood from the subject, (2) separating the blood components (e.g. based on density), and (3) returning certain component(s) of the blood to the subject by transfusion. The blood is normally separated into three fractions: red blood cells (about 45% of total blood), “buffy coat” (less than 1% of total blood) and plasma (about 55% of total blood). Various types of apheresis procedures can be used depending on the component of blood that is being removed.

Any methods including quantitative and qualitative methods can be used to identify that the hematopoietic stem cells have been mobilized into the peripheral blood. The methods typically involve isolating a quantity of the patient's blood and analyzing the quantity of the cells within the blood. Any method can be used to analyze the number of cells, including but not limited to: ELISA to identify the specific cells, FACS analysis, coulter counters and other blood counting devices, morphological identification, and PCR. The cells can be identified by any method known to one of skill in the art, including but not limited to, the identification of one or more proteins which are specifically expressed by the precursor cells, by morphology, by mRNA expression, and by PCR. The identification of the cells can be done at any time after administration of the agents (CXCR4 inhibitor which is administered following the natalizumab), included but not limited to: 5 minutes, 10 minutes, 20 minutes, 30 minutes, 45 minutes, 1 hour, 1.5 hours, 2 hours, 3 hours, 4 hours, 5 hours, 6 hours, 7 hours, 8 hours, 9 hours, 10 hours, 12 hours, 1 day, 2 days.

According to one embodiment, after collection (and optionally separation), the hematopoietic stem cells may be stored (e.g. for later use), expanded in culture or administered to a subject in need thereof (as discussed in detail herein below). Accordingly, the method of mobilizing stem cells can be used for mobilization of precursor hematopoietic cells in individuals who will serve as allogenic or autologous donors of hematopoietic stem cells.

In order to obtain sufficient number of hematopoietic precursor cells for transplantation, the collected hematopoietic stem cells may be further cultured in vitro in order to increase expansion.

During in vitro culturing, the hematopoietic precursor cells may change their cell phenotype. The presence or absence of an antigen on the surface of cells may be analyzed using specific antibodies (e.g. CD34 antibodies) by methods well known by the person skilled in the art, like, for example, ELISA of FACS. It may further be analyzed by other methods well known in the art, like RT-PCR or the like.

The hematopoietic precursor cells can additionally be used for gene therapy. Because pluripotent hematopoietic stem cells are self-renewing, and give rise to blood cell progenitors as well as mature blood cells, the hematopoietic precursor cells are an appropriate target for gene therapy. After collection (and optionally separation), the hematopoietic precursor cells can be modified to deliver gene products upon reintroduction to the individual. After modification, the cells are reinfused into a subject in need of such treatment.

According to another aspect of the invention, there is provided an isolated population of hematopoietic precursor cells obtainable by the method of some embodiments of the invention.

The phrase “isolated population of cells” as used herein refers to cells which have been isolated from their natural environment (e.g., the human body).

According to one embodiment, the population may comprise the CXCr4 inhibitor and/or natalizuman.

According to one embodiment, the cells are cultured ex vivo or in vitro.

Each of the agents can be administered to the subject per se or as part of a pharmaceutical composition which also includes a physiologically acceptable carrier. The purpose of a pharmaceutical composition is to facilitate administration of the active ingredient to an organism.

It will be appreciated that the pharmaceutical composition may further comprise other compounds such as described in detail hereinabove.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent which affects mobilization.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Dosage amount and interval may be adjusted individually to provide adequate levels of the active ingredient as to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Agents of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Typical conditions that can be ameliorated or otherwise benefited by enhanced hematopoietic stem cell survival and/or expansion and/or mobilization and/or hematopoietic precursor cell transplantation, include, but are not limited to, hematopoietic disorders and malignancies, such as aplastic anemia, lymphoma, leukemia, immune deficiency, severe combined immune deficiency (SCID), cytopenia (e.g. anemia, leukopenia, neutropenia, thrombocytopenia, granulocytopenia, pancytopenia), drug-induced cytopenia, toxin-induced cytopenia, radiation-induced cytopenia, cytopenia associated with conventional bone marrow transplantation, hematopoietic deficits associated with oncology treatment, with chemotherapy, or with radiation therapy, anemia or cytopenia of chronic disease (also referred to as anemia or cytopenia of chronic inflammation), osteopetrosis, Gaucher's disease, thalassemia and other congenital or genetically-determined hematopoietic abnormalities.

According to one embodiment, the methods of the invention are useful in enhancing the success of transplantation during and following immunosuppressive treatments as well as in effecting more efficient wound healing and treatment of bacterial inflammation.

According to one embodiment, the methods of the invention are useful for treating subjects who are immunocompromised or whose immune system is otherwise impaired. Typical conditions that are ameliorated or otherwise benefited by the method of the present invention include, but are not limited to, those subjects who are infected with a retrovirus and more specifically who are infected with human immunodeficiency virus (HIV).

Accordingly, the methods of the invention may be used for the treatment of a broad spectrum of conditions for which elevation of hematopoietic precursor cell levels in a subject would be beneficial or, where harvesting of precursor cell for subsequent stem cell transplantation would be beneficial. The compounds are also administered to regenerate myocardium by mobilizing bone marrow precursor cells.

The methods described herein are also particularly suitable for those subjects in need of repeated or high doses of chemotherapy. For some cancer patients, hematopoietic toxicity frequently limits the opportunity for chemotherapy dose escalation or completion of prescribed chemotherapy.

Repeated or high dose cycles of chemotherapy can be responsible for severe precursor cell depletion leading to important long-term hematopoietic sequelea and marrow exhaustion. The methods of the present invention provide for improved cell survival, blood cell reconstitution and blood cell count when used in conjunction with chemotherapy.

In one embodiment, the disease or condition is a malignant disease.

As used herein, the term “malignant disease” or “cancer” refers to any cancerous disease. Cancer cells may be associated with phenotypes such uncontrolled proliferation, loss of specialized functions, immortality, significant metastatic potential, significant increase in anti-apoptotic activity, rapid growth and proliferation rate, and certain characteristic morphology and cellular markers.

In some circumstances, cancer cells will be in the form of a tumor, such cells may exist locally within an animal (e.g. solid tumor), alternatively, cancer cells may circulate in the blood stream as independent cells, for example, leukemic cells (non-solid tumor), or may be dispersed throughout the body (e.g. metastasis). It will be appreciated that the term “cancer” as used herein encompasses all types of cancers, at any stage and in any form.

Types of malignant diseases amenable to diagnosis or treatment by the methods of some embodiments of the invention include benign tumors, warts, polyps, pre-cancers, and malignant tumors/cancers.

Specific examples of cancerous diseases which can be treated using the methods of the present invention include, but are not limited to, carcinoma, lymphoma, blastoma, sarcoma, and leukemia. More particular examples of such cancers include squamous cell cancer, soft-tissue sarcoma, Kaposi's sarcoma, melanoma, lung cancer (including small-cell lung cancer, non-small-cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, rectal cancer, endometrial or uterine carcinoma, carcinoid carcinoma, salivary gland carcinoma, kidney or renal cancer, liver cancer, prostate cancer, vulval cancer, thyroid cancer, hepatic carcinoma, mesothelioma, multiple myeloma, post-transplant lymphoproliferative disorder (PTLD), and various types of head and neck cancer (e.g. brain tumor). The cancerous conditions amenable for treatment of the invention include metastatic cancers.

According to one embodiment, the malignant disease is a hematological malignancy. Exemplary hematological malignancies include, but are not limited to, leukemia [e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia, T-cell acute lymphocytic leukemia (T-ALL) and B-cell chronic lymphocytic leukemia (B-CLL)] and lymphoma [e.g., Hodgkin's disease, non-Hodgkin's lymphoma, Burkitt, cutaneous T cell, histiocytic, lymphoblastic, T cell, thymic, B cell, including low grade/follicular; small lymphocytic (SL) NHL; intermediate grade/follicular NHL; intermediate grade diffuse NHL; high grade immunoblastic NHL; high grade lymphoblastic NHL; high-grade small non-cleaved cell NHL; bulky disease NHL; mantle cell lymphoma; AIDS-related lymphoma; and Waldenstrom's Macroglobulinemia].

According to a specific embodiment, the malignant disease is a leukemia, a lymphoma, a myeloma, a melanoma, a sarcoma, a neuroblastoma, a colon cancer, a colorectal cancer, a breast cancer, an ovarian cancer, an esophageal cancer, a synovial cell cancer, a hepatic cancer and a pancreatic cancer.

According to one embodiment, the subject has a non-malignant disease.

According to one embodiment, the non-malignant disease is an organ dysfunction or failure (e.g. renal disease or liver disease which are associated with cytopenia), a hematologic non-malignant disease (e.g. cytopenia, anemia, sickle cell disease, hemophilia), a graft related disease (e.g. graft rejection), an immune deficiency, a severe combined immunodeficiency syndromes (SCID), a genetic disease, a metabolic disorder, an infectious disease, an inflammatory disease (e.g. systemic inflammatory disease such as that associated with cytopenia), an autoimmune disease, an allergic disease, a trauma and an injury.
Inflammatory diseases—Include, but are not limited to, chronic inflammatory diseases and acute inflammatory diseases.
   Inflammatory Diseases Associated with Hypersensitivity

Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH.

Type I or immediate hypersensitivity, such as asthma.

Type II hypersensitivity include, but are not limited to, rheumatoid diseases, rheumatoid autoimmune diseases, rheumatoid arthritis (Krenn V. et al., Histol Histopathol 2000 July; 15(3):791), spondylitis, ankylosing spondylitis (Jan Voswinkel et al., Arthritis Res 2001; 3(3):189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6(2):156); Chan OT. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29(2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57(8):1810), myxedema, idiopathic myxedema (Mitsuma T. Nippon Rinsho. 1999 August; 57(8):1759); autoimmune reproductive diseases, ovarian diseases, ovarian autoimmunity (Garza K M. et al., J Reprod Immunol 1998 February; 37(2):87), autoimmune anti-sperm infertility (Diekman A B. et al., Am J Reprod Immunol. 2000 March; 43(3):134), repeated fetal loss (Tincani A. et al., Lupus 1998; 7Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante AJ. And Kraig E, Int Rev Immunol 1999; 18 (1-2):83), motor neuropathies (Kornberg AJ. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydenham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad Sci. 1998 May 13; 841:482), cardiovascular diseases, cardiovascular autoimmune diseases, atherosclerosis (Matsuura E. et al., Lupus. 1998; 7Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost.2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel LH. Ann Med Interne (Paris). 2000 May; 151 (3):178); antiphospholipid syndrome (Flamholz R. et al., J Clin Apheresis 1999; 14 (4):171); heart failure, agonist-like β-adrenoceptor antibodies in heart failure (Wallukat G. et al., Am J Cardiol. 1999 Jun. 17; 83 (12A):75H), thrombocytopenia purpura (Moccia F. Ann Ital Med Int. 1999 April-June; 14 (2):114); hemolytic anemia, autoimmune hemolytic anemia (Efremov DG. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Type IV or T cell mediated hypersensitivity, include, but are not limited to, rheumatoid diseases, rheumatoid arthritis (Tisch R, McDevitt HO. Proc Natl Acad Sci USA 1994 Jan. 18; 91 (2):437), systemic diseases, systemic autoimmune diseases, systemic lupus erythematosus (Datta SK., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopeni purpura (Semple J W. et al., Blood 1996 May 15; 87 (10):4245), anti-helper T lymphocyte autoimmunity (Caporossi AP. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones DE. Clin Sci (Colch) 1996 November; 91 (5):551), nephric diseases, nephric autoimmune diseases, nephritis, interstitial nephritis (Kelly CJ. J Am Soc Nephrol 1990 August; 1 (2):140), connective tissue diseases, ear diseases, autoimmune connective tissue diseases, autoimmune ear disease (Yoo TJ. et al., Cell Immunol 1994 August; 157 (1):249), disease of the inner ear (Gloddek B. et al., Ann N Y Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to, Tₕ1 lymphocyte mediated hypersensitivity and Tₕ2 lymphocyte mediated hypersensitivity.

### Autoimmune Diseases

Include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost.2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopeni purpura (Moccia F. Ann Ital Med Int. 1999 April-June; 14 (2):114; Semple J W. et al., Blood 1996 May 15; 87 (10):4245), autoimmune hemolytic anemia (Efremov DG. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt HO. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. Diseases include, but are not limited to autoimmune diseases of the pancreas, Type 1 diabetes (Castano L. and Eisenbarth GS. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns MP. J Hepatol 2000 August; 33 (2):326).

Examples of autoimmune neurological diseases include, but are not limited to, multiple sclerosis (Cross AH. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydenham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1): 92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo TJ. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan OT. et al., Immunol Rev 1999 June; 169:107).

### Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Specific types of viral pathogens causing infectious diseases treatable according to the teachings of the present invention include, but are not limited to, retroviruses, circoviruses, parvoviruses, papovaviruses, adenoviruses, herpesviruses, iridoviruses, poxviruses, hepadnaviruses, picornaviruses, caliciviruses, togaviruses, flaviviruses, reoviruses, orthomyxoviruses, paramyxoviruses, rhabdoviruses, bunyaviruses, coronaviruses, arenaviruses, and filoviruses.

Specific examples of viral infections which may be treated according to the teachings of the present invention include, but are not limited to, those caused by human immunodeficiency virus (HIV)-induced acquired immunodeficiency syndrome (AIDS), influenza, rhinoviral infection, viral meningitis, Epstein-Barr virus (EBV) infection, hepatitis A, B or C virus infection, measles, papilloma virus infection/warts, cytomegalovirus (CMV) infection, Herpes simplex virus infection, yellow fever, Ebola virus infection, rabies, Adenovirus (Adv), cold viruses, flu viruses, Japanese encephalitis, polio, respiratory syncytial, rubella, smallpox, varicella zoster, rotavirus, West Nile virus and zika virus.

Specific examples of bacterial infections which may be treated according to the teachings of the present invention include, but are not limited to, those caused by anthrax; gram-negative bacilli, chlamydia, diptheria, haemophilus influenza, Helicobacter pylori, malaria, Mycobacterium tuberculosis, pertussis toxin, pneumococcus, rickettsiae, staphylococcus, streptococcus and tetanus.

Specific examples of superbug infections (e.g. multi-drug resistant bacteria) which may be treated according to the teachings of the present invention include, but are not limited to, those caused by Enterococcus faecium, Clostridium difficile, Acinetobacter baumannii, Pseudomonas aeruginosa, and Enterobacteriaceae (including Escherichia coli, Klebsiella pneumoniae, Enterobacter spp.).

Specific examples of fungal infections which may be treated according to the teachings of the present invention include, but are not limited to, those caused by candida, coccidiodes, cryptococcus, histoplasma, leishmania, plasmodium, protozoa, parasites, schistosoma, tinea, toxoplasma, and Trypanosoma cruzi.

### Graft Rejection Diseases

According to other embodiment, the disease is associated with transplantation of a graft. Examples of diseases associated with transplantation of a graft include, but are not limited to, graft rejection, chronic graft rejection, subacute graft rejection, hyperacute graft rejection, acute graft rejection, allograft rejection, xenograft rejection and graft-versus-host disease (GVHD).

### Allergic Diseases

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

### Non-Malignant Hematologic Disease

Examples of non-malignant hematologic diseases include, but are not limited to, cytopenia (e.g. anemia, leukopenia, neutropenia, thrombocytopenia, granulocytopenia, pancytopenia), drug-induced cytopenia, toxin-induced cytopenia, radiation-induced cytopenia or cytopenia associated with conventional bone marrow transplantation, bone marrow disorders, deep vein thrombosis/pulmonary embolism, diamond blackfan anemia, hemochromatosis, hemophilia, immune hematologic disorders, iron metabolism disorders, sickle cell disease, thalassemia, osteopetrosis, Von Willebrand disease, and Gaucher's disease.

In addition, the method of mobilizing hematopoietic stem cells can be used for patients who are “difficult to mobilize” because, for example, they are not sensitive to growth factors.

The hematopoietic stem cells of the present invention may be transplanted into a recipient using any method known in the art for cell transplantation, such as but not limited to, cell infusion (e.g. I.V.) or via an intraperitoneal route.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of mobilizing hematopoietic precursor cells from the bone marrow to the peripheral blood of a subject, comprising:
(a) administering to the subject an effective amount of natalizumab; and
(b) administering to the subject an effective amount of a CXCR4 inhibitor, wherein step (b) is effected at least 48 hours following step (a).

2. A method for obtaining a therapeutically effective amount of hematopoietic precursor cells from a subject, comprising:
(a) mobilizing hematopoietic precursor cells from the bone marrow to the peripheral blood according to claim 1;
and subsequently
(b) harvesting said hematopoietic precursor cells by apheresis.

3-4. (canceled)

5. The method of claim 1, wherein said subject is a human subject.

6. The method of claim 1, wherein said subject is a donor of said hematopoietic precursor cells.

7. The method of claim 1, wherein the subject is diagnosed with cancer.

8. The method of claim 1, wherein said hematopoietic precursor cells are for autologous transplantation.

9. The method of claim 1, wherein said hematopoietic precursor cells are for allogeneic transplantation.

10. The method of claim 1, wherein said effective amount comprises repetitive cycles of administration of said natalizumab and said CXCR4 inhibitor.

11. The method of claim 1, wherein said at least 48 hours comprises up to 96 hours.

12. The method of claim 1, wherein said at least 48 hours comprises up to 72 hours.

13. The method of claim 1, wherein said CXCR4 inhibitor is a peptide, a small molecule, an antibody, a nucleic acid or a combination of same.

14. The method of claim 1, wherein said CXCR4 inhibitor is a peptide as set forth in SEQ ID NO: 1.

15. The method of claim 1, wherein said CXCR4 inhibitor is a small molecule.

16. The method of claim 1, wherein said CXCR4 inhibitor is AMD3100.


SEQUENCE LISTING (72 sequences)

SEQ ID NO: 1 - protein, 14 aa, synthetic construct
  REGION 1..14: note = synthetic peptide
  MOD_RES 1: note = 4-fluorobenzoyl-arginine
  MOD_RES 3: note = 3-(2-naphthyl) alanine
  MOD_RES 6: note = citrulline
  MOD_RES 8: note = D-lysine
  MOD_RES 12: note = citrulline
  MOD_RES 14: note = C' AMIDATED
RRACYRKKPYRRCR

SEQ ID NO: 2 - protein, 14 aa, synthetic construct
  REGION 1..14: note = SYNTHETIC PEPTIDE
  MOD_RES 1: note = N' ACETYLATION
  MOD_RES 3: note = 3-(2-naphthyl) alanine
  MOD_RES 6: note = citrulline
  MOD_RES 8: note = D-lysine
  MOD_RES 12: note = citrulline
RRACYRKKPYRRCR

SEQ ID NO: 3 - protein, 14 aa, synthetic construct
  REGION 1..14: note = SYNTHETIC PEPTIDE
  MOD_RES 1: note = N' ACETYLATION
  MOD_RES 3: note = 3-(2-naphthyl) alanine
  MOD_RES 8: note = D-citrulline
  MOD_RES 12: note = citrulline
RRACYRKRPYRRCR

SEQ ID NO: 4 - protein, 14 aa, synthetic construct
  REGION 1..14: note = synthetic peptide
  MOD_RES 1: note = N' ACETYLATED
  MOD_RES 3: note = 3-(2-naphthyl) alanine
  MOD_RES 6: note = citrulline
  MOD_RES 8: note = D-citrulline
  MOD_RES 12: note = citrulline
RRACYRKRPYRRCR

SEQ ID NO: 5 - protein, 14 aa, synthetic construct
  REGION 1..14: note = synthetic peptide
  MOD_RES 1: note = N' ACETYLATED
  MOD_RES 3: note = 3-(2-naphthyl) alanine
  MOD_RES 6: note = citrulline
  MOD_RES 8: note = D-lysine
  MOD_RES 11..12: note = citrulline
RRACYRKKPYRRCR

SEQ ID NO: 6 - protein, 14 aa, synthetic construct
  REGION 1..14: note = synthetic peptide
  MOD_RES 1: note = N' acetylated citrulline
  MOD_RES 3: note = 3-(2-naphthyl) alanine
  MOD_RES 6: note = citrulline
  MOD_RES 8: note = D-lysine
  MOD_RES 12: note = citrulline
RRACYRKKPYRRCR

SEQ ID NO: 7 - protein, 14 aa, synthetic construct
  REGION 1..14: note = synthetic peptide
  MOD_RES 1: note = N' acetylated citrulline
  MOD_RES 3: note = 3-(2-naphthyl) alanine
  MOD_RES 8: note = D-citrulline
  MOD_RES 12: note = citrulline
RRACYRKRPYRRCR

SEQ ID NO: 8 - protein, 14 aa, synthetic construct
  REGION 1..14: note = synthetic peptide
  MOD_RES 1: note = N' ACETYLATED
  MOD_RES 3: note = 3-(2-naphthyl) alanine
  MOD_RES 8: note = D-citrulline
  MOD_RES 11..12: note = citrulline
RRACYRKRPYRRCR

SEQ ID NO: 9 - protein, 14 aa, synthetic construct
  REGION 1..14: note = synthetic peptide
  MOD_RES 1: note = N' ACETYLATED citrulline
  MOD_RES 3: note = 3-(2-naphthyl) alanine
  MOD_RES 8: note = D-lysine
  MOD_RES 11..12: note = citrulline
RRACYRKKPYRRCR

SEQ ID NO: 10 - protein, 14 aa, synthetic construct
  REGION 1..14: note = synthetic peptide
  MOD_RES 1: note = N' ACETYLATED
  MOD_RES 3: note = 3-(2-naphthyl) alanine
  MOD_RES 6: note = citrulline
  MOD_RES 8: note = D-citrulline
  MOD_RES 12: note = citrulline
  MOD_RES 14: note = C' AMIDATED
RRACYRKRPYRRCR

SEQ ID NO: 11 - protein, 14 aa, synthetic construct
  REGION 1..14: note = synthetic peptide
  MOD_RES 1: note = N' ACETYLATED
  MOD_RES 3: note = 3-(2-naphthyl) alanine
  MOD_RES 6: note = citrulline
  MOD_RES 8: note = D-lysine
  MOD_RES 11..12: note = citrulline
  MOD_RES 14: note = C' AMIDATED
RRACYRKKPYRRCR

SEQ ID NO: 12 - protein, 14 aa, synthetic construct
  REGION 1..14: note = synthetic peptide
  MOD_RES 1: note = N' ACETYLATED citrulline
  MOD_RES 3: note = 3-(2-naphthyl) alanine
  MOD_RES 6: note = citrulline
  MOD_RES 8: note = D-lysine
  MOD_RES 12: note = citrulline
  MOD_RES 14: note = AMIDATED
RRACYRKKPYRRCR

SEQ ID NO: 13 - protein, 14 aa, synthetic construct
  REGION 1..14: note = synthetic peptide
  MOD_RES 1: note = N' ACETYLATED citrulline
  MOD_RES 3: note = 3-(2-naphthyl) alanine
  MOD_RES 8: note = D-citrulline
  MOD_RES 12: note = citrulline
  MOD_RES 14: note = C' AMIDATED
RRACYRKRPYRRCR

SEQ ID NO: 14 - protein, 14 aa, synthetic construct
  REGION 1..14: note = synthetic peptide
  MOD_RES 1: note = N' ACETYLATED
  MOD_RES 3: note = 3-(2-naphthyl) alanine
  MOD_RES 8: note = D-citrulline
  MOD_RES 11..12: note = citrulline
  MOD_RES 14: note = C' AMIDATED
RRACYRKRPYRRCR

SEQ ID NO: 15 - protein, 14 aa, synthetic construct
  REGION 1..14: note = synthetic peptide
  MOD_RES 1: note = N' ACETYLATED citrulline
  MOD_RES 3: note = 3-(2-naphthyl) alanine
  MOD_RES 8: note = D-lysine
  MOD_RES 11..12: note = citrulline
  MOD_RES 14: note = C' AMIDATED
RRACYRKKPYRRCR

SEQ ID NO: 16 - protein, 14 aa, synthetic construct
  REGION 1..14: note = synthetic peptide
  MOD_RES 1: note = D-glutamic acid
  MOD_RES 3: note = 3-(2-naphthyl) alanine
  MOD_RES 8: note = D-lysine
  MOD_RES 12: note = citrulline
ERACYRKKPYRRCR

SEQ ID NO: 17 - protein, 14 aa, synthetic construct
  REGION 1..14: note = synthetic peptide
  MOD_RES 3: note = 3-(2-naphthyl) alanine
  MOD_RES 8: note = D-lysine
  MOD_RES 12: note = citrulline
REACYRKKPYRRCR

SEQ ID NO: 18 - protein, 14 aa, synthetic construct
  REGION 1..14: note = synthetic peptide
  MOD_RES 3: note = 3-(2-naphthyl) alanine
  MOD_RES 8: note = D-lysine
  MOD_RES 12: note = citrulline
RRACYEKKPYRRCR

SEQ ID NO: 19 - protein, 14 aa, synthetic construct
  REGION 1..14: note = synthetic peptide
  MOD_RES 3: note = 3-(2-naphthyl) alanine
  MOD_RES 8: note = D-lysine
  MOD_RES 12: note = citrulline
RRACYREKPYRRCR

SEQ ID NO: 20 - protein, 14 aa, synthetic construct
  REGION 1..14: note = synthetic peptide
  MOD_RES 3: note = 3-(2-naphthyl) alanine
  MOD_RES 8: note = D-glutamic acid
  MOD_RES 12: note = citrulline
RRACYRKEPYRRCR

SEQ ID NO: 21 - protein, 14 aa, synthetic construct
  REGION 1..14: note = synthetic peptide
  MOD_RES 3: note = 3-(2-naphthyl) alanine
  MOD_RES 8: note = D-lysine
  MOD_RES 12: note = citrulline
RRACYRKKPYERCR

SEQ ID NO: 22 - protein, 14 aa, synthetic construct
  REGION 1..14: note = synthetic peptide
  MOD_RES 3: note = 3-(2-naphthyl) alanine
  MOD_RES 8: note = D-lysine
  MOD_RES 12: note = citrulline
RRACYRKKPYRRCE

SEQ ID NO: 23 - protein, 14 aa, synthetic construct
  REGION 1..14: note = synthetic peptide
  MOD_RES 3: note = 3-(2-naphthyl) alanine
  MOD_RES 6: note = citrulline
  MOD_RES 8: note = D-glutamic acid
  MOD_RES 12: note = citrulline
  MOD_RES 14: note = C' amidated
RRACYRKEPYRRCR

SEQ ID NO: 24 - protein, 14 aa, synthetic construct
  REGION 1..14: note = synthetic peptide
  MOD_RES 3: note = 3-(2-naphthyl) alanine
  MOD_RES 6: note = D-glutamic acid
  MOD_RES 8: note = D-citrulline
  MOD_RES 12: note = citrulline
  MOD_RES 14: note = c' amidated
RRACYEKRPYRRCR

SEQ ID NO: 25 - protein, 14 aa, synthetic construct
  REGION 1..14: note = synthetic peptide
  MOD_RES 3: note = 3-(2-naphthyl) alanine
  MOD_RES 6: note = D-glutamic acid
  MOD_RES 8: note = D-glutamic acid
  MOD_RES 12: note = citrulline
  MOD_RES 14: note = c' amidated
RRACYEKEPYRRCR

SEQ ID NO: 26 - protein, 14 aa, synthetic construct
  REGION 1..14: note = synthetic peptide
  MOD_RES 1: note = D-glutamic acid
  MOD_RES 3: note = 3-(2-naphthyl) alanine
  MOD_RES 6: note = citrulline
  MOD_RES 8: note = D-glutamic acid
  MOD_RES 12: note = citrulline
  MOD_RES 14: note = c' amidated
ERACYRKEPYRRCR

SEQ ID NO: 27 - protein, 14 aa, synthetic construct
  REGION 1..14: note = synthetic peptide
  MOD_RES 3: note = 3-(2-naphthyl) alanine
  MOD_RES 6: note = citrulline
  MOD_RES 8: note = D-glutamic acid
  MOD_RES 10: note = D-glutamic acid
  MOD_RES 12: note = citrulline
  MOD_RES 14: note = c' amidated
RRACYRKEPERRCR

SEQ ID NO: 28 - protein, 14 aa, synthetic construct
  REGION 1..14: note = synthetic peptide
  MOD_RES 3: note = 3-(2-naphthyl) alanine
  MOD_RES 6: note = citrulline
  MOD_RES 8: note = D-glutamic acid
  MOD_RES 12: note = D-glutamic acid
  MOD_RES 14: note = c' amidated
RRACYRKEPYRECR

SEQ ID NO: 29 - protein, 14 aa, synthetic construct
  REGION 1..14: note = synthetic peptide
  MOD_RES 1: note = N' ACETYLATED D-glutamic acid
  MOD_RES 3: note = 3-(2-naphthyl) alanine
  MOD_RES 6: note = citrulline
  MOD_RES 8: note = D-glutamic acid
  MOD_RES 12: note = citrulline
  MOD_RES 14: note = c' amidated
ERACYRKEPYRRCR

SEQ ID NO: 30 - protein, 14 aa, synthetic construct
  REGION 1..14: note = synthetic peptide
  MOD_RES 1: note = N' ACETYLATED
  MOD_RES 3: note = 3-(2-naphthyl) alanine
  MOD_RES 6: note = citrulline
  MOD_RES 8: note = D-glutamic acid
  MOD_RES 10: note = D-glutamic acid
  MOD_RES 12: note = citrulline
  MOD_RES 14: note = c' amidated
RRACYRKEPERRCR

SEQ ID NO: 31 - protein, 14 aa, synthetic construct
  REGION 1..14: note = synthetic peptide
  MOD_RES 1: note = N' ACETYLATED
  MOD_RES 3: note = 3-(2-naphthyl) alanine
  MOD_RES 6: note = citrulline
  MOD_RES 8: note = D-glutamic acid
  MOD_RES 12: note = D-glutamic acid
  MOD_RES 14: note = c' amidated
RRACYRKEPYRECR

SEQ ID NO: 32 - protein, 14 aa, synthetic construct
  REGION 1..14: note = synthetic peptide
  MOD_RES 1: note = N' ACETYLATED
  MOD_RES 3: note = 3-(2-naphthyl) alanine
  MOD_RES 6: note = citrulline
  MOD_RES 8: note = D-glutamic acid
  MOD_RES 12: note = citrulline
  MOD_RES 14: note = c' amidated
RRACYRKEPYRRCR

SEQ ID NO: 33 - protein, 14 aa, synthetic construct
  REGION 1..14: note = synthetic peptide
  MOD_RES 1: note = Guanyl-arginine
  MOD_RES 3: note = 3-(2-naphthyl) alanine
  MOD_RES 6: note = citrulline
  MOD_RES 8: note = D-glutamic acid
  MOD_RES 12: note = citrulline
  MOD_RES 14: note = c' amidated
RRACYRKEPYRRCR

SEQ ID NO: 34 - protein, 14 aa, synthetic construct
  REGION 1..14: note = synthetic peptide
  MOD_RES 1: note = Tetramethylguanyl-arginine
  MOD_RES 3: note = 3-(2-naphthyl) alanine
  MOD_RES 6: note = citrulline
  MOD_RES 8: note = D-glutamic acid
  MOD_RES 12: note = citrulline
  MOD_RES 14: note = c' amidated
RRACYRKEPYRRCR

SEQ ID NO: 35 - protein, 13 aa, synthetic construct
  REGION 1..13: note = synthetic peptide
  MOD_RES 1: note = Tetramethylguanyl-arginine
  MOD_RES 2: note = 3-(2-naphthyl) alanine
  MOD_RES 5: note = citrulline
  MOD_RES 7: note = D-glutamic acid
  MOD_RES 11: note = citrulline
  MOD_RES 13: note = c' amidated
RACYRKEPYRRCR

SEQ ID NO: 36 - protein, 14 aa, synthetic construct
  REGION 1..14: note = synthetic peptide
  MOD_RES 1: note = 4-fluorobenzoyl-arginine
  MOD_RES 3: note = 3-(2-naphthyl) alanine
  MOD_RES 6: note = citrulline
  MOD_RES 8: note = D-glutamic acid
  MOD_RES 12: note = citrulline
  MOD_RES 14: note = amidated
RRACYRKEPYRRCR

SEQ ID NO: 37 - protein, 14 aa, synthetic construct
  REGION 1..14: note = synthetic peptide
  MOD_RES 1: note = 2-fluorobenzoyl-arginine
  MOD_RES 3: note = 3-(2-naphthyl) alanine
  MOD_RES 6: note = citrulline
  MOD_RES 8: note = D-glutamic acid
  MOD_RES 12: note = citrulline
  MOD_RES 14: note = c' amidated
RRACYRKEPYRRCR

SEQ ID NO: 38 - protein, 13 aa, synthetic construct
  REGION 1..13: note = synthetic peptide
  MOD_RES 1: note = 5-aminopentanoyl-arginine
  MOD_RES 2: note = 3-(2-naphthyl) alanine
  MOD_RES 5: note = citrulline
  MOD_RES 7: note = D-glutamic acid
  MOD_RES 11: note = citrulline
  MOD_RES 13: note = c' amidated
RACYRKEPYRRCR

SEQ ID NO: 39 - protein, 14 aa, synthetic construct
  REGION 1..14: note = synthetic peptide
  MOD_RES 1: note = 2-desamino-arginyl
  MOD_RES 3: note = 3-(2-naphthyl) alanine
  MOD_RES 6: note = citrulline
  MOD_RES 8: note = D-glutamic acid
  MOD_RES 12: note = citrulline
  MOD_RES 14: note = c' amidated
RRACYRKEPYRRCR

SEQ ID NO: 40 - protein, 13 aa, synthetic construct
  REGION 1..13: note = synthetic peptide
  MOD_RES 1: note = Guanyl-arginine
  MOD_RES 2: note = 3-(2-naphthyl) alanine
  MOD_RES 5: note = citrulline
  MOD_RES 7: note = D-glutamic acid
  MOD_RES 11: note = citrulline
  MOD_RES 13: note = c' amidated
RACYRKEPYRRCR

SEQ ID NO: 41 - protein, 13 aa, synthetic construct
  REGION 1..13: note = synthetic peptide
  MOD_RES 1: note = Succinyl-arginine
  MOD_RES 2: note = 3-(2-naphthyl) alanine
  MOD_RES 5: note = citrulline
  MOD_RES 7: note = D-glutamic acid
  MOD_RES 11: note = citrulline
  MOD_RES 13: note = c' amidated
RACYRKEPYRRCR

SEQ ID NO: 42 - protein, 13 aa, synthetic construct
  REGION 1..13: note = synthetic peptide
  MOD_RES 1: note = Glutaryl-arginine
  MOD_RES 2: note = 3-(2-naphthyl) alanine
  MOD_RES 5: note = citrulline
  MOD_RES 7: note = D-glutamic acid
  MOD_RES 11: note = citrulline
  MOD_RES 13: note = c' amidated
RACYRKEPYRRCR

SEQ ID NO: 43 - protein, 13 aa, synthetic construct
  REGION 1..13: note = synthetic peptide
  MOD_RES 1: note = desaminoTMG-APA conjugated
  MOD_RES 2: note = 3-(2-naphthyl) alanine
  MOD_RES 5: note = citrulline
  MOD_RES 7: note = D-glutamic acid
  MOD_RES 11: note = citrulline
  MOD_RES 13: note = c' amidated
RACYRKEPYRRCR

SEQ ID NO: 44 - protein, 13 aa, synthetic construct
  REGION 1..13: note = synthetic peptide
  MOD_RES 1: note = R-CH2 -conjugated ( formula (V) in the specification)
  MOD_RES 3: note = 3-(2-naphthyl) alanine
  MOD_RES 5: note = citrulline
  MOD_RES 7: note = D-glutamic acid
  MOD_RES 11: note = citrulline
  MOD_RES 13: note = c' amidated
RACYRKEPYRRCR

SEQ ID NO: 45 - protein, 13 aa, synthetic construct
  REGION 1..13: note = synthetic peptide
  MOD_RES 2: note = 3-(2-naphthyl) alanine
  MOD_RES 5: note = citrulline
  MOD_RES 7: note = D-glutamic acid
  MOD_RES 11: note = citrulline
  MOD_RES 13: note = c' amidated
RACYRKEPYRRCR

SEQ ID NO: 46 - protein, 14 aa, synthetic construct
  REGION 1..14: note = synthetic peptide
  MOD_RES 1: note = tetramethylguanyl-arginine
  MOD_RES 3: note = 3-(2-naphthyl) alanine
  MOD_RES 6: note = citrulline
  MOD_RES 8: note = D-citrulline
  MOD_RES 12: note = citrulline
  MOD_RES 14: note = C' AMIDATED
RRACYRKRPYRRCR

SEQ ID NO: 47 - protein, 14 aa, synthetic construct
  REGION 1..14: note = synthetic peptide
  MOD_RES 1: note = 6-aminohexanoyl-arginine
  MOD_RES 3: note = 3-(2-naphthyl) alanine
  MOD_RES 6: note = citrulline
  MOD_RES 8: note = D-citrulline
  MOD_RES 12: note = citrulline
  MOD_RES 14: note = C' AMIDATED
RRACYRKRPYRRCR

SEQ ID NO: 48 - protein, 14 aa, synthetic construct
  REGION 1..14: note = synthetic peptide
  MOD_RES 1: note = 6-aminohexanoyl-arginine
  MOD_RES 3: note = 3-(2-naphthyl) alanine
  MOD_RES 8: note = D-lysine
  MOD_RES 12: note = citrulline
RRACYRKKPYRRCR

SEQ ID NO: 49 - protein, 14 aa, synthetic construct
  REGION 1..14: note = synthetic peptide
  MOD_RES 3: note = 3-(2-naphthyl) alanine
  MOD_RES 6: note = citrulline
  MOD_RES 8: note = D-lysine
  MOD_RES 12: note = citrulline
  MOD_RES 14: note = C' amidated
RRACYRRKPYRRCR

SEQ ID NO: 50 - protein, 14 aa, synthetic construct
  REGION 1..14: note = synthetic peptide
  MOD_RES 1: note = N' ACETYLATED
  MOD_RES 3: note = 3-(2-naphthyl) alanine
  MOD_RES 6: note = citrulline
  MOD_RES 8: note = D-lysine
  MOD_RES 12: note = citrulline
  MOD_RES 14: note = C' amidated
RRACYRRKPYRRCR

SEQ ID NO: 51 - protein, 14 aa, synthetic construct
  REGION 1..14: note = SYNTHETIC PEPTIDE
  MOD_RES 1: note = N' ACETYLATED
  MOD_RES 3: note = 3-(2-naphthyl) alanine
  MOD_RES 6: note = citrulline
  MOD_RES 8: note = D-lysine
  MOD_RES 12: note = citrulline
  MOD_RES 14: note = C' AMIDATED
RRACYRKKPYRRCR

SEQ ID NO: 52 - protein, 14 aa, synthetic construct
  REGION 1..14: note = synthetic peptide
  MOD_RES 1: note = N' ACETYLATED
  MOD_RES 3: note = 3-(2-naphthyl) alanine
  MOD_RES 8: note = D-citrulline
  MOD_RES 12: note = citrulline
  MOD_RES 14: note = C' AMIDATED
RRACYRKRPYRRCR

SEQ ID NO: 53 - protein, 14 aa, synthetic construct
  REGION 1..14: note = synthetic peptide
  MOD_RES 1: note = 4-fluorobenzoyl-arginine
  MOD_RES 3: note = 3-((2-naphthyl) alanine
  MOD_RES 6: note = citrulline
  MOD_RES 8: note = D-glutamic acid
  MOD_RES 12: note = citrulline
  SITE 14: note = MISC_FEATURE - derivatization by a NH-methyl group
RRACYRKEPYRRCR

SEQ ID NO: 54 - protein, 14 aa, synthetic construct
  REGION 1..14: note = synthetic peptide
  MOD_RES 1: note = 4-fluorobenzoyl-arginine
  MOD_RES 3: note = 3-((2-naphthyl) alanine
  MOD_RES 6: note = citrulline
  MOD_RES 8: note = D-glutamic acid
  MOD_RES 12: note = citrulline
  SITE 14: note = MISC_FEATURE - derivatization by a NH-ethyl group
RRACYRKEPYRRCR

SEQ ID NO: 55 - protein, 14 aa, synthetic construct
  REGION 1..14: note = synthetic peptide
  MOD_RES 1: note = 4-fluorobenzoyl-arginine
  MOD_RES 3: note = 3-((2-naphthyl) alanine
  MOD_RES 6: note = citrulline
  MOD_RES 8: note = D-glutamic acid
  MOD_RES 12: note = citrulline
  SITE 14: note = MISC_FEATURE - derivatization by NH-isopropyl
RRACYRKEPYRRCR

SEQ ID NO: 56 - protein, 14 aa, synthetic construct
  REGION 1..14: note = synthetic peptide
  MOD_RES 1: note = 4-fluorobenzoyl-arginine
  MOD_RES 3: note = 3-((2-naphthyl) alanine
  MOD_RES 6: note = citrulline
  MOD_RES 8: note = D-glutamic acid
  MOD_RES 12: note = citrulline
  SITE 14: note = MISC_FEATURE - derivatization with a tyramine residue
RRACYRKEPYRRCR

SEQ ID NO: 57 - protein, 14 aa, synthetic construct
  REGION 1..14: note = synthetic peptide
  MOD_RES 3: note = 3-((2-naphthyl) alanine
  MOD_RES 8: note = D-lysine
  MOD_RES 12: note = citrulline
ARACYRKKPYRRCR

SEQ ID NO: 58 - protein, 14 aa, synthetic construct
  REGION 1..14: note = synthetic peptide
  MOD_RES 3: note = 3-((2-naphthyl) alanine
  MOD_RES 8: note = D-lysine
  MOD_RES 12: note = citrulline
RRACYAKKPYRRCR

SEQ ID NO: 59 - protein, 14 aa, synthetic construct
  REGION 1..14: note = synthetic peptide
  MOD_RES 3: note = 3-((2-naphthyl) alanine
  MOD_RES 8: note = D-lysine
  MOD_RES 12: note = citrulline
RRACYRAKPYRRCR

SEQ ID NO: 60 - protein, 14 aa, synthetic construct
  REGION 1..14: note = synthetic peptide
  MOD_RES 3: note = 3-((2-naphthyl) alanine
  MOD_RES 8: note = D-alanine
  MOD_RES 12: note = citrulline
RRACYRKAPYRRCR

SEQ ID NO: 61 - protein, 14 aa, synthetic construct
  REGION 1..14: note = synthetic peptide
  MOD_RES 3: note = 3-((2-naphthyl) alanine
  MOD_RES 8: note = D-lysine
  MOD_RES 12: note = citrulline
RRACYRKKAYRRCR

SEQ ID NO: 62 - protein, 14 aa, synthetic construct
  REGION 1..14: note = synthetic peptide
  MOD_RES 3: note = 3-((2-naphthyl) alanine
  MOD_RES 8: note = D-lysine
  MOD_RES 12: note = citrulline
RRACYRKKPARRCR

SEQ ID NO: 63 - protein, 14 aa, synthetic construct
  REGION 1..14: note = synthetic peptide
  MOD_RES 3: note = 3-((2-naphthyl) alanine
  MOD_RES 8: note = D-lysine
  MOD_RES 12: note = citrulline
RRACYRKKPYARCR

SEQ ID NO: 64 - protein, 14 aa, synthetic construct
  REGION 1..14: note = synthetic peptide
  MOD_RES 1: note = citrulline
  MOD_RES 3: note = 3-((2-naphthyl) alanine
  MOD_RES 8: note = D-lysine
  MOD_RES 12: note = citrulline
RRACYRKKPYRRCR

SEQ ID NO: 65 - protein, 14 aa, synthetic construct
  REGION 1..14: note = synthetic peptide
  MOD_RES 3: note = 3-((2-naphthyl) alanine
  MOD_RES 6: note = citrulline
  MOD_RES 8: note = D-lysine
  MOD_RES 12: note = citrulline
RRACYRKKPYRRCR

SEQ ID NO: 66 - protein, 14 aa, synthetic construct
  REGION 1..14: note = synthetic peptide
  MOD_RES 3: note = 3-((2-naphthyl) alanine
  MOD_RES 6: note = citrulline
  MOD_RES 8: note = D-lysine
  MOD_RES 12: note = citrulline
  MOD_RES 14: note = C' AMIDATED
RRACYRKKPYRRCR

SEQ ID NO: 67 - protein, 14 aa, synthetic construct
  REGION 1..14: note = synthetic peptide
  MOD_RES 3: note = 3-((2-naphthyl) alanine
  MOD_RES 7: note = citrulline
  MOD_RES 8: note = D-lysine
  MOD_RES 12: note = citrulline
RRACYRRKPYRRCR

SEQ ID NO: 68 - protein, 14 aa, synthetic construct
  REGION 1..14: note = synthetic peptide
  MOD_RES 3: note = 3-((2-naphthyl) alanine
  MOD_RES 6: note = citrulline
  MOD_RES 8: note = D-citrulline
  MOD_RES 12: note = citrulline
  MOD_RES 14: note = C' AMIDATED
RRACYRKRPYRRCR

SEQ ID NO: 69 - protein, 14 aa, synthetic construct
  REGION 1..14: note = synthetic peptide
  MOD_RES 3: note = 3-((2-naphthyl) alanine
  MOD_RES 8: note = D-lysine
  MOD_RES 12: note = citrulline
RRACYRKKPYRRCR

SEQ ID NO: 70 - protein, 14 aa, synthetic construct
  REGION 1..14: note = synthetic peptide
  MOD_RES 3: note = 3-((2-naphthyl) alanine
  MOD_RES 6: note = citrulline
  MOD_RES 8: note = D-citrulline
  MOD_RES 12: note = citrulline
RRACYRKRPYRRCR

SEQ ID NO: 71 - protein, 14 aa, synthetic construct
  REGION 1..14: note = synthetic peptide
  MOD_RES 3: note = 3-((2-naphthyl) alanine
  MOD_RES 8: note = D-citrulline
  MOD_RES 12: note = citrulline
RRACYRKRPYRRCR

SEQ ID NO: 72 - protein, 14 aa, synthetic construct
  REGION 1..14: note = synthetic peptide
  MOD_RES 1: note = citrulline
  MOD_RES 3: note = 3-((2-naphthyl) alanine
  MOD_RES 8: note = D-citrulline
  MOD_RES 12: note = citrulline
  MOD_RES 14: note = C' Amidated
RRACYRKRPYRRCR